(19) United States
(12) Patent Application Publication
(10) Pub. No.: US 2016/0025759 A1
(43) Pub. Date: Jan. 28, 2016

(54) APPARATUS AND METHODS FOR IDENTIFYING THE FRONT AND THE REAR OF A TEST BOARD

(71) Applicant: ABON BIOHPARM (HANGZHOU) CO., LTD, Hangzhou (CN)

(72) Inventors: Kang YANG, Hangzhou (CN); Jingliu ZHANG, Hangzhou (CN)

(21) Appl. No.: 14/774,128

(22) PCT Filed: Mar. 22, 2014
(86) PCT No.: PCT/CN2014/073914

§ 371 Date: Sep. 9, 2015

(30) Foreign Application Priority Data
Apr. 3, 2013 (CN) 201310116639.1
Apr. 3, 2013 (CN) 201320165529.X

(51) Int. Cl. G01N 35/04; G01N 33/53

(52) U.S. Cl. CPC G01N 35/04; G01N 33/53; G01N 2035/0494

(57) ABSTRACT

This invention relates to an apparatus that could identify the front and the rear of a test board, comprising a support supporting the test board and a filter block on the support, wherein the filter block will not be in contact with test boards in the first direction, which makes them move along the first route; and it will be in contact with test boards in the second direction, which makes them move along the second route which is different from the first route. In the meantime, this invention also comprises the methods to identify the front and back of a test board with this apparatus. By using apparatus and methods in the present invention to identify and sort test boards, it will improve the efficiency in mechanical assembly of test boards and reduce the cost.

### CROSS REFERENCE

This invention claims the prior Chinese applications with the filing No. 201310116639.1 and 201320165529.X, both of which are filed on 3 Apr., 2013.

### FIELD OF THE INVENTION

The present invention relates to an identifying apparatus and identifying methods, particularly to an apparatus and methods for identifying the front and the rear of a test board with test strips.

### BACKGROUND OF THE INVENTION

Immune binding reaction principle is used to test if there is targeted substance in samples. This technology has been widely used in various fields to test the targeted substance in multiple biological samples (saliva, blood, urine, serum and sweat, etc) to monitor illness and human health condition (early pregnancy, tumor, infectious disease and drug, etc). A plurality of such tests can be finished on solid substrate material, such as common lateral flow solid substrate material, glass or plastic porous plate and immunochromatography apparatus, etc. In some tests, to be convenient for the operators' self-detection, test strips to be detected are arranged on the test board and corresponding test strips are arranged on the same test board especially when the same sample is used to detect a plurality of ingredients at the same time.

Generally, such test boards are manually operated with low efficiency and high cost. Therefore, automated assembly with machine is tried in current assembly manufacturing to improve the efficiency and reduce the human cost.

How to make test boards sorted to the same side and the same direction on equipment is a very critical issue in assembly operation of test boards with equipment. In some embodiments, a vibrating disk is used to vibrate and drop test boards onto the subsequent conveyer belt one after another, wherein test boards are randomly arranged on the vibrating track, conveyed onto the belt and people will remove test boards in the wrong sort order and the wrong direction after observation. In some other embodiments, a sensing device is used to detect the side and the direction of test boards when they fall onto the conveyer belt with incorrect test boards being taken away by mechanical arms. In such embodiments, the sorting of test boards is passive with low efficiency and generally will affect efficiency of the overall test board assembly equipment, and will also increase the production cost with additional equipment in the production line.

### SUMMARY OF THE INVENTION

The present invention relates to an apparatus that could automatically identify and sort test boards when assembling test boards with a machine or equipment. In particularly, when using a vibrating disk to assemble test boards, this apparatus can sort test boards facing the same side or orientation to the same direction and automatically remove those test boards in the wrong direction so that all the test boards in the same direction can proceed to the next working procedure in order with operating efficiency improved.

This invention relates to an apparatus that could identify the front and the rear of a test board, comprising a support supporting the test board and a filter block on the support, wherein the filter block will not be contact with test boards in the first direction, which makes them move along the first route; and it will be contact with test boards in the second direction, which makes them move along the second route which is different from the first route. That is to say, when a test board is moving on the support in the first direction, the test board will not be in contact with the filter block and it will continue moving along the support in the original route (the first route); and when a test board is moving on the support in the second direction, the test board will be in contact with the filter block and it will change route, deviate from the support and move along a new route (the second route).

In the present invention, the filter block on the support is used to differentiate test boards in opposite directions. How to achieve it? Specific plan in the present invention is to use the filter block to selectively contact with test boards moving on the support in different directions so as to tell them apart. To be specific, the filter block will not be contact with test boards in the first direction, which makes them move along the first route; and it will be contact with test boards in the second direction, which makes them move along the second route. Or, the filter block will be contact with test boards in the first direction, which makes them move along the second route; and it will not be contact with test boards in the second direction, which makes them move along the first route, wherein the first route is the scheduled moving route for test boards and the second route is the new route generated due to the test board's contact with the filter block and deviation from the support. That is to say, when the test board moves forward on the support in the first direction, it will not be in contact with the filter block on the support and it will move along the scheduled route on the support; however, when the test board moves forward in the second direction, it will be contact with the filter block which will generate resistance and change its moving direction and route on the support, which means the test board will deviate from the support and move along the new route.

In a specific embodiment, the front and the rear of a test board is identified via the filter block's contact or no contact with the support pin on it. In such way, test boards in different directions can be separated and those in the same direction can move along the same direction with others changing their moving direction so as to realize the sorting and separation of test boards in the same direction.

In a more specific embodiment, the filter block is not in contact with the support pin on test boards in the first direction, which makes them move along the first route; and the filter block is in contact with the support pin on test boards in the second direction, which changes their direction to move along the second route.

In some preferred embodiments, test boards are moving along the same route before passing the filter block no matter they are in the first or second direction. After they pass the filter block, test boards in different directions are separated by the filter block: test boards in the same direction (such as the first direction) can move along the same direction (the first route); and those test boards in opposite or different direction are removed and move along the second route. In a specific program of the present invention, this is realized based on the filter block's contact or no contact with the protruding apparatus—the support pin on the test board. To be specific, if the support pin is not in contact with the filter block when the test board faces one direction, they must contact with each other when the test board faces the other opposite direction; vice versa, if the support pin is in contact with the filter block when the test board faces one direction, they must not contact with each other when the test board faces the other opposite direction, wherein the term “support pin” is positioned on the test board. In this way, two test boards in opposite directions can be differentiated. In an embodiment, the support pin is arranged at reverse side of the test board and is assembled facing the test strip, raising from inner surface of the test board and forming a raised structure. The bulge could be formation of the support pin and can be twig or rib shape, or cylinder, cuboid, cube or combination of the abovementioned shapes, etc.

To be more specific, the test board is positioned on the support and can move forward along the support wall with a filter block fastening on the support like a protruding structure, which means all the test boards will pass the said filter block when moving on the support; besides, a support pin is arranged on the test board at the side in contact with the support; when moving on the support, the support pin on the test board may selectively have contact with the said filter block, which is to say, make support pins on test boards in one direction to get in contact or no contact with the filter block and make support pins on test boards in the other direction to correspondingly get no contact or contact with the filter block so that test boards in the same one direction can be reserved and others removed. In a specific embodiment, the support pin on the test board is not in contact with the filter block when the test board is in the first direction and it is in contact with the filter block when the test board is in the second direction. To be more specific, when the test board in the first direction moves on the support, it will not be in contact with the filter block and will move forward on the support along the scheduled route; however, when the test board in the second direction moves on the support, it will be in contact with the filter block, slow down due to the resistance, change the route, deviate from the support and move along a new route. It is further preferred that the test board with direction changed can fall off the support and for example, drop onto the vibrating disk bottom so that they can be re-vibrated onto the support for screening. In some preferred embodiments, test boards in the first direction could be those test boards with sample loading holes in the front and reading windows at the rear, and correspondingly, test boards in the second direction could be those test boards with reading windows at the rear and sample loading holes in the front.

In another embodiment, the front and the rear of the test board are identified when the filter block is positioned under two or more than two intervals with different width on the test board.

In more specific embodiments, the filter block is designed to not be in contact with the test board when the filter block is positioned under a wide interval of the test board which will move along the first route; and the filter block is designed to be in contact with the test board when the filter block is positioned under a narrow interval of the test board which will deviate from the support and move along the second route. It can also be understood in the way that size of the filter block can be designed so that some test boards passing by change their direction and some don't change after intervals with different width on test boards are determined. For instance, test boards moving along the first direction all the time will not change their direction, but test boards moving along the second direction will change their direction so as to differentiate them. Size of the filter block could be less than the wide interval so that the entire filter block can pass the interval with no contact with the test board when passing the wide interval. However, part of the filter block is less than the narrow interval and part of it is greater than the narrow interval, in which way, when passing the narrow interval, the narrow part of the filter block can first get into the narrow interval for further movement and the wide part of the filter block can be in contact with the component forming the narrow interval so as to change the direction. These test boards with direction changed are removed and the others with direction unchanged are reserved.

In some embodiments, intervals are segmented by support pins on test boards. It is preferred that support pins segment two or more than two intervals with different width on test boards and different distances among them. When the test board passes the filter block, the wide interval in one direction is not in contact with the filter block and will not change the direction; when the narrow interval passes the filter block, the filter block will be in contact with components constituting the interval like the support pin, and the test board moving forward will change its direction due to resistance so as to move towards the same direction as test boards without contact with the filter block. In such circumstance, screening and sorting can be achieved. In some preferred embodiments, two intervals with different width are segmented by 3 support pins.

In the present invention, the filter block can also be used to differentiate two opposite directions of test boards based on different intervals on them by making the filter block not in contact with the test board when passing the wide interval on the test board which will move along the original route, and making the filter block in contact with the test board when passing the narrow interval on the test board which will change direction and move along a new route. In this embodiment, the wide interval is made to pass the filter block when the test board in the first direction is moving, and the narrow interval is made to pass the filter block when the test board in the second direction is moving on the support.

It is certainly optional. In the present invention, the filter block can also be used to differentiate two opposite directions of test boards based on different intervals on them by making the filter block not in contact with the test board when passing the narrow interval on the test board which will move along the original route, and making the filter block in contact with the test board when passing the wide interval on the test board which will change direction and move along a new route.

In some embodiments, intervals are segmented by support pins on test boards. It is preferred that support pins segment two or more than two intervals with different width on test boards and different distances among them. If there are two and more than two intervals with the same width on test boards, in a preferred embodiment, the support pin of the test board is positioned in one of the intervals.

In a specific embodiment, when the test board is in the first direction, an interval without a support pin passes the filter block and the test board will move along the scheduled route; and when the test board is in the second direction, an interval with a support pin passes the filter block and the test board will change direction, deviate from the support pin and move along a new route.

In some specific embodiments, more than one test strips can be positioned on the test board with one or more than one intervals to isolate test strips. Generally speaking, these intervals are provided with the same width and distance with test strip information observation windows in the middle of the intervals. When the test board moves on the support, the filter block on the support is positioned as one of the intervals' moving above the filter block. In case there is no obstacle in the interval, the test board will pass the filter block and move forward along the support; however, if there is obstacle in the interval, such obstacle will be in contact with the filter block when the test board passes the filter block, under which circumstance, the test board will change its moving direction due to resistance between the filter block and the obstacle so as to deviate from the original route. Consequently, when the test board is in the first direction, the interval without a support pin will pass the filter block and the test board will continue moving along the scheduled route; and when the test board is in the second direction, the interval with a support pin will pass the filter block with the support pin being in contact with the filter block and the test board will change its direction due to resistance between the support pin and the filter block, deviate from the original route and the support. In this way, test boards in two different directions (the first direction and the second direction) can be differentiated.

In some specific embodiments, to effectively differentiate test boards in opposite directions, the filter block is in triangular block structure and the width of its bottom or its longest edge is less than or equal to the interval width of the test board. In a specific embodiment, the width of the bottom of the triangle is 1-2 mm.

In more specific embodiments, the hypotenuse of the filter block is in contact with the support pin of the test board. When the test board passes the filter block, the vertex of the triangular filter block first gets into the interval and then the entire filter block, in which way, the support pin will move from the vertex along the hypotenuse when the filter block is in contact with the support pin so that the test board can deviate from the support in a certain direction and move along the deflected route.

To make sure test boards in the first direction can move along the scheduled route, in more specific embodiments, the height of the filter block is less than the height of the support pin since the test board is in contact with the support at the side with a support pin, and the distance from the test board to the support is the height of the support pin. In this way, the filter block can be entirely under the test board without contact with other part of it so that the test board can move normally without resistance from the filter block. In a specific embodiment, the support pin of the test board is 5 mm high and the filter block is 1-4 mm high.

In a preferred embodiment, the width of the support is the same or substantially the same as the width of the test board so that the test board can move smoothly along the scheduled route.

In another preferred embodiment, the identifying apparatus in the present invention also comprises a component removing test boards from the support.

In more specific embodiments, the said component is positioned at the outer edge of the support, corresponding to the outer edge of the test board deviated from the support.

In a specific embodiment, the component comprises an air pipe above the support, air blown from which can blow test boards in wrong direction away from the support. It is preferred that the blown test boards can fall into the bottom of the apparatus (the vibrating disk or the vibrating pan) and get onto the support of the apparatus randomly again for screening and sorting.

In another specific embodiment, the said component comprises a bulge above the edge of the deviated test board, which will compress the test board to make it fall off the support when the test board deviates from the support to the bulge.

Filter block in the present invention differentiates test boards in opposite directions. However, in assembly equipment, to be convenient for the next working procedure, it is generally required to make all the test boards in the same orientation, which means test boards in the same direction are required. Therefore, test boards in the other direction had better to be removed after being differentiated. In some preferred embodiments, the component removing unsatisfactory test boards in the other direction is also included to further remove deviated test boards away from the support. In specific embodiments, the said component is an air pipe above the test board, air blown from which can blow test boards in the opposite direction, i.e. test boards in wrong direction, away from the support. In more specific embodiments, the said air pipe is positioned at the edge of the support, especially the support-free edge on the deviated test board, in which way, air pressure blown from the air pipe can act upon this place to smoothly remove the test board away from the support.

In another aspect, the present invention also comprises the methods for identifying the front and the rear of the test board: make the test board move along the support and pass the filter block on the support, the filter block will not be contact with the test board in the first direction, which makes it move along the first route; and it will be contact with the test board in the second direction, which makes it move along the second route which is different from the first route.

In a preferred embodiment, the filter block is not in contact with the support pin on the test board in the first direction, which makes it move along the first route; and it is in contact with the test board in the second direction, which makes it change its direction and move along the second route.

In a specific embodiment, the test board comprises more than one intervals and the support pin is arranged in one of the intervals.

In more specific embodiments, when the test board is in the first direction, an interval without a support pin passes the filter block and the test board moves along the scheduled route; when the test board is in the second direction, an interval with a support pin passes the filter block and the test board changes its direction, deviates from the support pin and moves along a new route.

### Beneficial Effects

By using the apparatus and methods in the present invention to identify and sort test boards, test boards can be sorted to the same direction, which improves the efficiency of the test board assembly with machine and reduces the cost.

### BRIEF DESCRIPTION OF THE DRAWINGS

FIG. 1 shows the schematic equipment view including the apparatus in the present invention which is usually in a pan shape;

FIG. 2 shows the schematic front view of a test board in the present invention;

FIG. 3 shows the schematic back view of a test board in the present invention;

FIG. 4a and FIG. 4b show the schematic view of another test board in the present invention, wherein FIG. 4a shows the test board with the front upside and the rear downside, and FIG. 4b shows the test board with the front downside and the rear upside, which show the same test board in different directions;

FIG. 5a and FIG. 5b show the schematic view of another test board in the present invention, wherein FIG. 5a shows the test board with the front upside and the rear downside, and FIG. 5b shows the test board with the front downside and the rear upside, which show the same test board in different directions;

FIG. 6a shows the schematic view of the route (the first route) when the test board in the first direction passes the filter block with direction unchanged;

FIG. 6b shows the schematic view of the route (the second route) when the test board in the second direction passes the filter block with direction changed;

FIG. 7 to FIG. 9 show the schematic view of the route for the test board's moving on the support in the identifying apparatus in the present invention;

FIG. 10 and FIG. 11 show the schematic view of the other route for the test board's moving on the support in the identifying apparatus in the present invention;

### BRIEF DESCRIPTION OF MARKS ON THE DRAWINGS

An apparatus 10 identifying both sides of a test board; an apparatus 20 identifying different directions of a test board; an equipment 30 comprising the two apparatuses; a support wall 200; a test board 100; a test board window 101; test board intervals 102, 1021, 1022, 1023, 1024, 1025, 1026, 1027; a support 700; support pins 104, 1041,1042 on the test board; the test board front end 109; the test board rear end 110; a filter block 600; a filter block vertex 601; the first route 701; the second route 702; an air pipe 800; and an air pipe outlet 801.

### Description of the Preferred Embodiment

Structure or technical terms involved in the present invention will be further illustrated next. In the following detailed description, reference text for the legend is part of it and examples will be given to describe possible specific program to be executed in the present invention. We do not rule out other specific programs and we might change and improve the present invention under the circumstance of not violating the essence of the present invention.

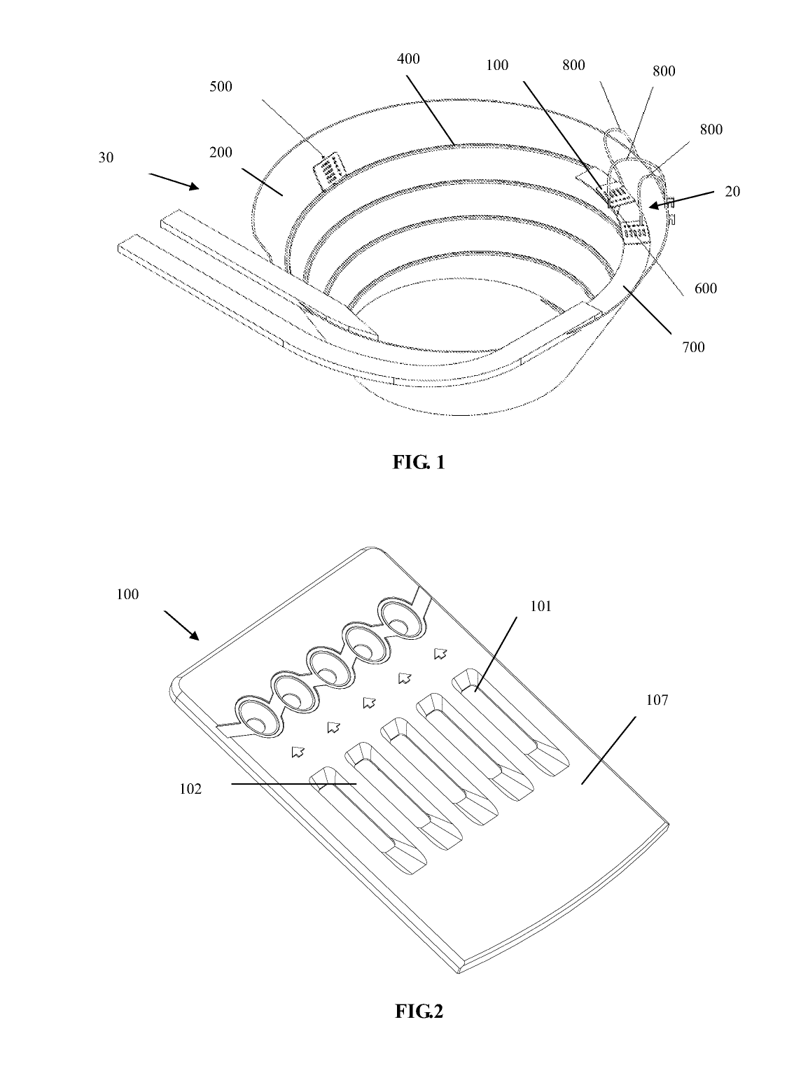

For example, FIG. 1 shows an equipment 30 used to sort the test board 100 to the same side and the same direction, and the apparatus 10 identifying both sides of a test board is applied on such equipment 30, being able to make such equipment 30 sort test boards to the same side. At the same time, on the equipment 30, there is also an apparatus 20 arranging test boards to the same direction which could sort test boards facing the same side to the same direction again, or sort them independently. The apparatus 10 and the apparatus 20 could make the equipment 30 sort test boards to the same side and the same direction and proceed to the next working procedure. In the meanwhile, they can improve the efficiency of the equipment 30.

The equipment 30 is generally a vibrating apparatus, like a vibrating disk that was dsicripted in published Chinese application, like ZL201220595118.X, 201120106726.5 and 201110091811.3, which randomly arranges messy test boards on the vibrating track without any rule on the direction and the orientation of the test boards, which needs to be improved to make such random test boards sorted uniformly in the consistent direction and orientation on the track. That is to say, test boards finally moving on the track are the same (the front upside or downside) with the front end in the front and the rear end at the rear, and those inconsistent test boards are removed in advance. Furthermore, test boards are moving on the track via vibration and the track of the vibratory equipment is generally static with test boards moving forward under vibration. Consequently, the support 700 and the filter block fastened on the support 700 are usually stationary.

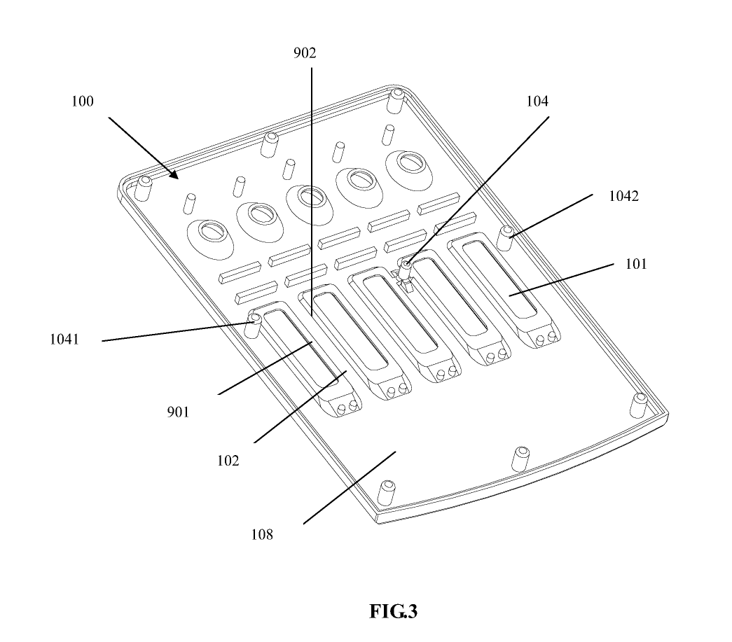

In general, the test board for assembly is made up of the upper test board and the lower test board with a test strip arranged on the lower board and the upper test board put on the lower test board, forming the final test reagent board or the testing apparatus. In a specific embodiment, the part of the test board assembled with the present invention as shown in FIG. 2 and FIG. 3, is the upper test board, wherein FIG. 2 shows the schematic front view of the upper test board 100 and FIG. 3 shows the schematic back view of the upper test board 100. The said test board 100 is made up of plates and there is a window 101 displaying the test strip result and an interval 102 between windows. At the edge of the test board, there is a protruding support pin 1042, assorted with another test board (the lower test board); meanwhile, there are another 2 support pins 104 and 1041, the distance between which is different from the distance between the support pin 104 and the support pin 1042, for instance, the former distance is longer and the latter distance is shorter.

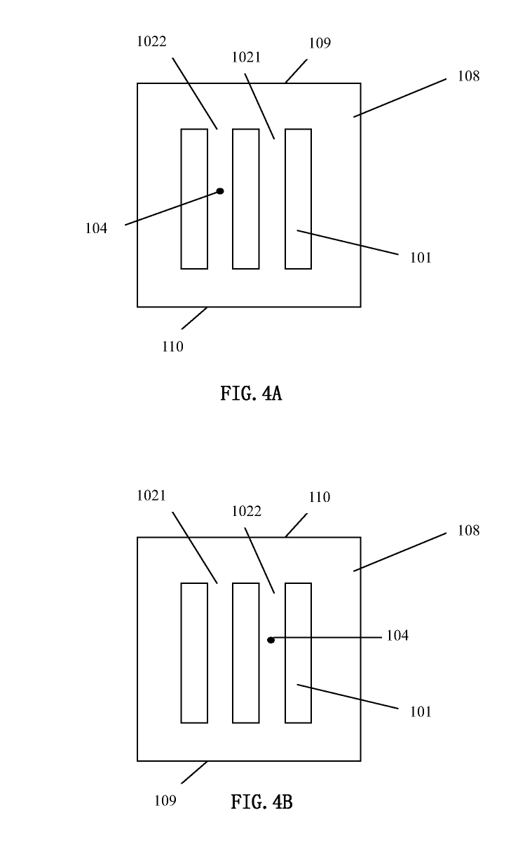

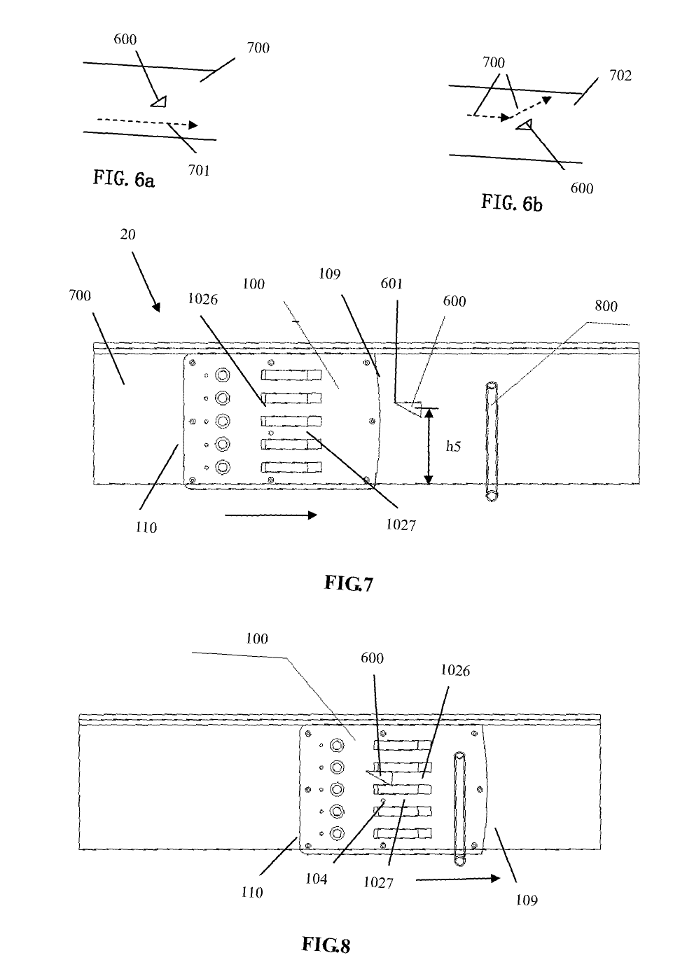

The interval here can also consist of the protruding fillet 901 and 902 on the reverse concave side 108. In FIG. 3, there are many such intervals with test strip result reading windows between them. The interval is formed by the side edge of the protruding fillet 901 and 902 into a “groove” shape with a support pin in the groove. If the support pin is positioned in one of the grooves, the filter block 600 is positioned to not pass the groove with a support pin when the test board in one direction (the first direction) passes the filter block 600, and the filter block 600 is positioned to pass the groove with a support pin when the test board in the other direction (the direction opposite to or different from the first direction) passes the filter block 600, in which way the filter block will be in contact with the support pin and the test board will change its direction. In the present invention, the apparatus for identifying the front and the rear of a test board also comprises a support 700 and a filter block 600 to differentiate the front and the rear of the test board 100 on the support, wherein the filter block 600 is fastened on the support 700 and in the moving route of the test board 100. As shown in FIG. 4a, the filter block 600 is not in contact with the test board 100 when the test board 100 in the first direction passes the filter block 600, in which way the test board 100 will move on the support 700 along the original route, i.e. a straight line 701 (FIG. 6a) (taking 109 as the front end and 110 as the rear end for forward direction as an example). However, they will be in contact with each other when the test board 100 in the second direction (the same test board with different direction, the rear end 110 in the front and the front end 109 at the rear for the reverse direction as an example) passes the filter block 600. Besides, due to resistance generated by the stationary filter block 600 upon the moving test board 100, the test board 100 changes its direction along the route in contact with the filter block (such as the hypotenuse of the triangle filter block) and forms a new moving route 702 as shown in FIG. 6b.

In some specific embodiment, the said filter block 600 is in triangular block structure so that it can differentiate the test board 100 in different directions in a better way, and the vertex 601 of the filter block triangle first gets into the test board 100 when it moves and passes the filter block 600.

Similarly, to make sure the test board 100 in one direction can smoothly pass the said filter block 600, in a specific embodiment, the height of the projection (i.e. the height of the filter block) of the said filter block 600 on the support 700 is no greater than the maximum thickness of the test board 100 and all components on it. On the test board 100, there is a support pin 104 which can identify direction of the test board in combination with the filter block 600, and the position of the support pin 104 on the test board 100 as well as the position of the filter block 600 on the support 700 shall meet the following requirements: when the test board 100 moves on the support 700 towards one direction, it can smoothly pass the filter block 600; however, if the test board moves on the support 700 towards the other opposite direction, it can be blocked by the filter block 600, which means the support pin 104 on the test board is blocked by the filter block 600. Such test boards being blocked cannot move along the support 700 and the others continue to move forward so that the test board 100 in two opposite directions can be differentiated. To giving fill play to its role of blocking the test board 100, the filter block 600 gets in contact with the support pin 104 via its hypotenuse, in which way, the test board 100 can change its direction based on the hypotenuse direction and deviate from the support 700.

The test board 100 applicable to the apparatus 20 can be provided with two and more than two intervals 102. In an embodiment, as shown in FIG. 4a and FIG. 4b, there are 2 intervals 1021 and 1022 on the test board 100, being symmetrical with the support pin 104 in one interval 1022. When the front end 109 of the test board is in the front and moves on the support 700 of the apparatus 20, as shown in FIG. 4a, one side 108 of the test board (the side 108 with support pins) is in contact with the support 700. When the test board 100 moves to the filter block 600 fastened on the support, the filter block 600 is positioned in the interval 1021. At this time, the width of the overall filter block is less than the width of the interval 1021 and the height of the filter block is less than the height of the test board 100 or the interval thickness (the height of the support pin plus the thickness of the test board at this time), and the test board 100 can smoothly pass the filter block 600; however, when the rear end 110 of the test board is in the front (the same test board but in different direction) and moves on the support 700, as shown in FIG. 4b, since the interval 1021 is symmetrical to the interval 1022, the interval 1022 with a support pin 104 on the support 700 is positioned at the former position of the interval 1021 so that the interval 1022 is positioned on the filter block 600 and the support pin 104 in the interval 1022 is in contact with the filter block 600 when the test board 100 passes the filter block 600 with the resistance generated changing the moving direction of the test board 100.

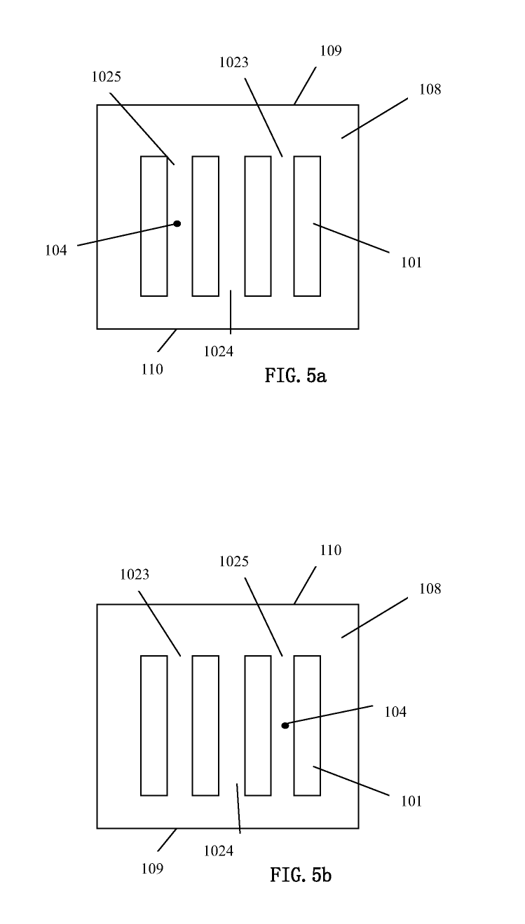

In another embodiment, as shown in FIG. 5a and FIG. 5b, there are 3 intervals 1023, 1024 and 1025 on the test board 100 with the interval 1023 being symmetrical to the interval 1025, wherein there is a support pin 104 in the interval 1025 and no support pin in the interval 1023. When the front end 109 of the test board is in the front and moves on the support 700, as shown in FIG. 5a, the filter block 600 is under the interval 1023; and when the rear end 110 of the test board is in the front and moves on the support 700, as shown in FIG. 5b, the filter block 600 is under the interval 1025 and is in contact with the support pin 104. In these two embodiments, the test board 100 smoothly passes the filter block 600 when the test board is in one direction; however, when the test board 100 is in the other direction, the support pin 104 will be contact with the filter block 600 and the test board 100 cannot pass the filter block 600 so that test boards in two directions can be differentiated. By such method, when there is more than one interval 102 on the test board and the test board is on the support, the support pin 104 is required to be positioned in another symmetrical interval if the filter block 600 is in one of the intervals.

Next, a specific test board identifying apparatus will be described with test boards to be differentiated as shown in FIG. 2 and FIG. 3. The test board 100 is provided with the capacity of 5 test strips and 5 windows with 4 intervals 102 among the windows in the same distance and same interval width. Therefore, the first interval is symmetrical to the fourth interval and the second interval 1026 is symmetrical to the third interval 1027 with the support pin 104 in the third interval 1027. As shown in FIG. 7, the support 700 and the test board 100 have the same width of 50 mm and there is a stationary filter block 600 with the height of 3 mm on the support 700; the width of the bottom of the filter block triangle is 2 mm and the vertex 601 of the filter block faces the moving direction of the test board. The position of the filter block 600 on the support 700 is determined by the position of the support 104, which means the filter block 600 is required to be in contact with the support pin 104 when the rear end of the test board faces forwards on the support 700. In this embodiment, the width of the test board is 50 mm and the distance from the support pin center to one edge of the test board is 20 mm and the distance to the other edge is 30 mm. As a result, the distance h5 from the filter block center to the outer edge of the support 700 is 30 mm as shown in FIG. 12.

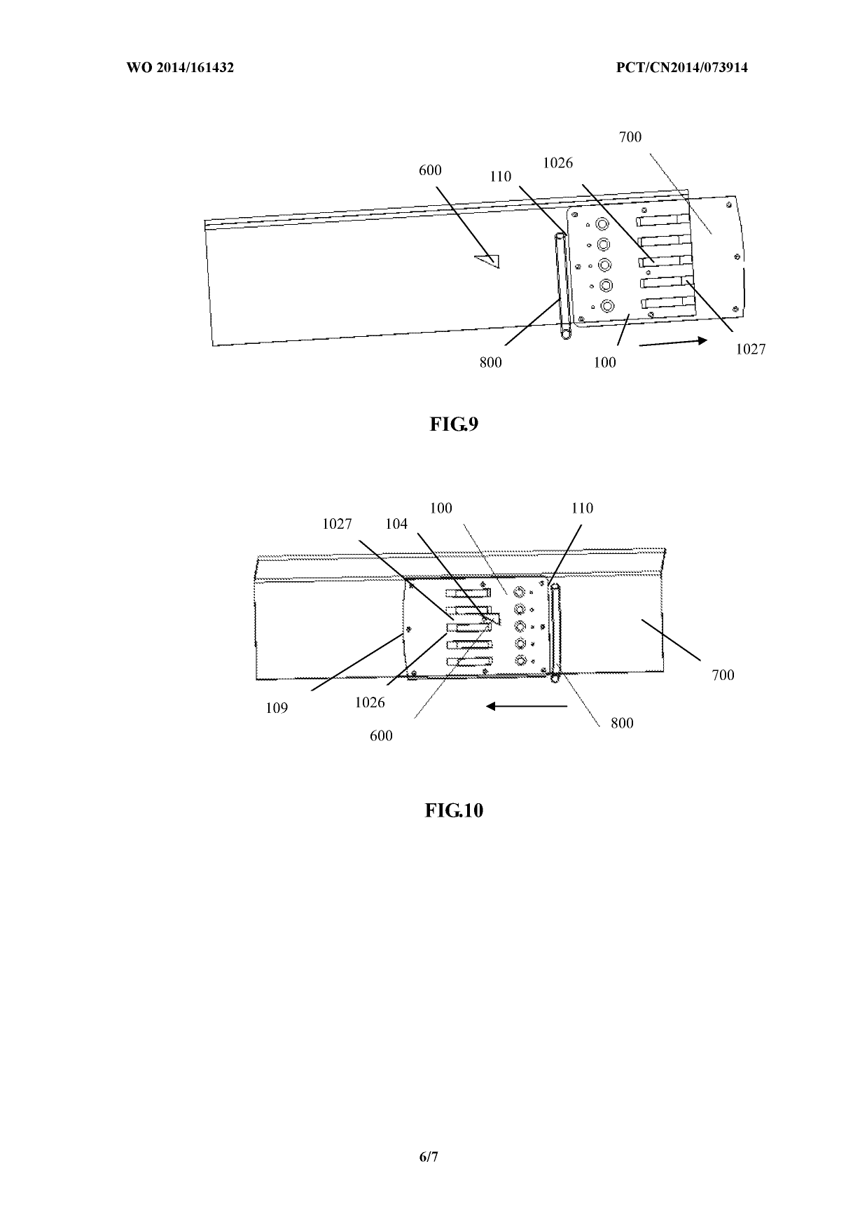

FIG. 7 to FIG. 9 show the schematic view of the moving of the test board 100 (the front end facing forwards) in the right direction on the support 700. As shown in FIG. 7, when the test board 100 in the right direction is on the support 700 with the front end 109 of the test board in the front and the rear end 110 at the rear, it will move towards the filter block 600. It can be seen from FIG. 7 that the position of the filter block 600 is corresponding to the second interval 1026 on the test board 100 at this time. When the test board 100 moves right above the filter block 600, as shown in FIG. 8, the vertex 601 of the triangle filter block will first get into the second interval 1026 and then the overall filter block 600, smoothly passing the second interval 1026. Then, after the test board 100 passes the filter block 600, it still moves along the support 700 as shown in FIG. 14 until the next working procedure.

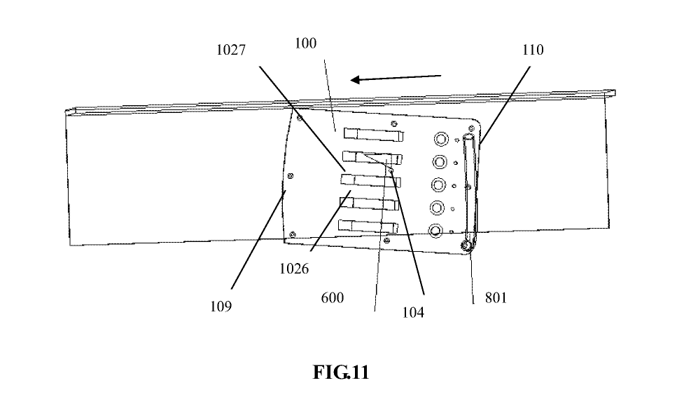

However, FIG. 10 and FIG. 11 show the schematic view of the moving of the test board 100 (rear end facing forwards) in the wrong direction on the support 700. When the test board 100 moves on the support 700 with the rear end 110 in the front and the front end 109 at the rear, as shown in FIG. 10, the filter block 600 is corresponding to the third interval 1027 on the test board, i.e. the interval with a support pin 104. Moreover, as shown in FIG. 10, when the test board 100 in this direction moves to the filter block 600, the vertex 601 of the filter block will get into the third interval 1027 and get in contact with the support pin 104 in such interval during its continuous movement, being blocked by the bevel of the filter block 600 so as to deviate from the outside of the support as shown in FIG. 11. Thereby, such test board 100 cannot continue to move along the support 700.

In a more specific embodiment, the said apparatus 20 identifying the direction of a test board also comprises another air pipe 800 to provide air and air pressure with the outlet 801 of such air pipe being positioned above and behind the filter block 600, and to be more specific, above the test board 100 deviated due to the filter block 600. As shown in FIG. 11, to be more specific, the outlet 801 of such air pipe is positioned at the edge of the deviated test board 100 from the support. In this way, when air blows such part of the test board, it will be pressed downwards due to the air pressure and fall off since there is no support here, so that the entire test board 100 will fall off from the support 700. All test boards 100 in this direction will break away from the support 700 and test boards 100 in the other direction will be reserved on the support 700 to be convenient for operation in the next working procedure.

Two intervals with different width on the test board are segmented by support pins on it. Taking FIG. 3 as an example, the distance from the support pin 104 to the support pin 1041 is different from the distance from the support pin 104 to the support pin 1042, and the former is greater than the latter. The filter block is designed in such way that it will not be in contact with any component on the test board, especially the support pin, when passes the support pin 1041 and 104 so as to not change the direction; and it will be in contact with the support pin when passes the support pin 104 and 1042 so as to change the moving direction (FIG. 3).

In the above embodiments, there could be a test strip reading window between support pins or no such window. The test boards is only taken as an example here to describe how to carry out the present invention and other components like a pen, a mobile phone and a card can also be sorted with the present invention.

1. An apparatus for identifying the front and the rear of a test board, comprising:
a support supporting the test board and a filter block on the support, wherein the filter block will not be contact with test boards in the first direction, which makes them move along the first route; and it will be contact with test boards in the second direction, which makes them move along the second route which is different from the first route.

2. The apparatus according to claim 1, wherein the said support is stationary relative to the test board.

3. The apparatus according to claim 1, wherein the front and the rear of a test board is identified based on if it is in contact with the filter block.

4. The apparatus according to claim 3, wherein the front and the rear of a test board is identified based on if the support pin on it is in contact with the filter block.

5. The apparatus according to claim 4, wherein the test board will move along the first route when the filter block is not in contact with the support pin on the test board in the first direction, and the test board will change direction and move along the second route when the filter block is in contact with the support pin on the test board in the second direction.

6. The apparatus according to claim 5, wherein test boards are moving to the same direction before they are in contact with the filter block.

7. The apparatus according to claim 1, wherein the front and the rear of a test board will be identified when the filter block is positioned under two or more than two intervals with different width on the test board.

8. The apparatus according to claim 7, wherein the filter block will not be in contact with the test board which will move along the first route when the filter block is positioned under a wide interval of the test board; and the filter block will be in contact with the test board which will deviate from the support and move along the second route when the filter block is positioned under a narrow interval of the test board.

9. The apparatus according to claim 8, wherein intervals are segmented by one or more than one support pins, and the said filter block's contact with the test board is realized via its contact with the support pin, and the said filter block's no contact with the test board is realized via its no contact with the support pin.

10. The apparatus according to claim 8, wherein test boards are moving to the same direction before they are in contact with the filter block.

11. The apparatus according to one of claim 7 10 claim 7, wherein the said filter block comprises a wide part and a narrow part and overall width of the filter block is less than the wide interval, with the width of the filter block's narrow part being less than the narrow interval and the width of the filter block's wide part being greater than the narrow interval.

12. The apparatus according to one of claim 11, wherein the filter block is in triangular block structure and its maximum width is less than or equal to the interval width on the test board.

13. The apparatus according to claim 11, wherein the narrow part of the filter block will first get into the wide interval or the narrow interval on the test board when the test board passes the filter block.

14. The apparatus according to claim 3, wherein there are two and more intervals with the same width on the test board and the said support pin is positioned in one interval and no support pin in the other one.

15. The apparatus according to claim 14, wherein an interval without a support pin will pass the filter block and the test board will move along the scheduled direction when the test board moves in the first direction; and the interval with a support pin will pass the filter block and the test board will change direction, deviate from the support pin and move along a new route when the test board moves in the second direction.

16. The apparatus according to claim 15, wherein test boards are moving to the same direction before they are in contact with the filter block.

17. The apparatus according to claim 7, wherein the height of the filter block is less than the height of the support pin.
